(19) United States
(12) Patent Application Publication
(10) Pub. No.: US 2026/0294417 A1
(43) Pub. Date: Oct. 1, 2026

(54) LEFT ATRIAL APPENDAGE OCCLUSION

(71) Applicants: Medtronic, Inc., Minneapolis, MN (US); Medtronic CryoCath LP, Toronto (CA)

(72) Inventors: Nicolas Coulombe, Anjou (CA); John B. Horrigan, Beverly, MA (US); Zhongping Yang, Woodbury, MN (US)

(21) Appl. No.: 19/479,279

(22) PCT Filed: Apr. 23, 2024
(86) PCT No.: PCT/IB2024/053942

§ 371 Date: Oct. 27, 2025

Related U.S. Application Data
(60) Provisional application No. 63/462,549, filed on Apr. 28, 2023.

(51) Int. Cl. A61B 17/00; A61B 90/00

(52) U.S. Cl. CPC A61B 17/0057; A61B 17/00234; A61B 90/06; A61B 90/39; A61B 2017/00022; A61B 2017/00243; A61B 2017/00305; A61B 2017/00557; A61B 2017/00623; A61B 2017/00632; A61B 2017/0065; A61B 2017/00951; A61B 2090/064; A61B 2090/3966

(57) ABSTRACT

A medical system, device and method are described for occluding a left atrial appendage. An example medical device, such as an occlusion device, may include a first catheter with a proximal end and a distal end. The distal end may be arranged to be positioned at an opening of the left atrial appendage or advanced into the left atrial appendage. A central lumen extends from a proximal portion to a distal portion of the first catheter to a deployable member arranged at the distal end of the first catheter. The deployable member may contact a wall of the atrial appendage and be retracted to at least partially invert the left atrial appendage, thus reducing its volume. A second catheter within the central lumen of the first catheter may be arranged to deliver adhesive into the left atrial appendage via the distal end of the first catheter.

This application is an international application claiming priority to U.S. provisional patent application No. 63/462,549, filed 28 Apr. 2023, the entire contents of which is incorporated herein by reference.

### FIELD OF THE INVENTION

The present invention relates to a method of occluding a left atrial appendage and an occlusion device for occluding a left atrial appendage.

### BACKGROUND OF THE INVENTION

The human heart is a four chambered, muscular organ that provides blood circulation through the body during a cardiac cycle. The four main chambers include the right atrium RA and right ventricle RV which supplies the pulmonary circulation, and the left atrium LA and left ventricle LV which supplies oxygenated blood received from the lungs into systemic circulation. To ensure that blood flows in one direction through the heart, atrioventricular valves (tricuspid valves TV and mitral valves MV) are present between the junctions of the atria and the ventricles, and semi-lunar valves (pulmonary valve and aortic valve) govern the exits of the ventricles leading to the lungs and the rest of the body.

The heart also includes a left atrial appendage LAA, which is a small, ear-shaped sac in the muscle wall of the left atrium LA. In normal hearts, when the heart contracts, the blood in the left atrium LA and the left atrial appendage LAA is squeezed out of the left atrium LA and into the left ventricle LV. The LAA has minimal influence on cardiac output and is generally considered to be a non-functional structure in the heart.

In atrial fibrillation, an irregular heartbeat causes blood flow to slow enabling clots to form. Because the left atrial appendage LAA is a small sac or pouch, blood may collect there and form clots. The clots often cause thromboembolic complications and risk to the patient. This risk increases if the thrombus or fragments of the thrombus dislodge. If all or a portion of the thrombus flows downstream, it is highly likely that the free material will become trapped in smaller and more tortuous anatomy. This increases an individual's risk for cerebral stroke or peripheral embolism. Thus, in some cases, it may be desirable to exclude or occlude the left atrial appendage LAA such that clots do not form in the left atrial appendage LAA, and if they do, they cannot escape the left atrial appendage LAA.

To reduce the above-mentioned risks, it is desirable to close the left-atrial appendage to reduce the occurrence of thrombus formation and the risk of thromboembolism. The present disclosure relates to improvements in catheter-based occlusion systems for occluding or excluding the left atrial appendage.

### SUMMARY OF THE INVENTION

A first aspect of the invention provides a method of occluding a left atrial appendage, the method comprising: providing a catheter with an elongate tubular body and a central lumen that extends from a proximal end to a distal end of the catheter, the catheter comprising a deployable member arranged at the distal end of the catheter; positioning the catheter adjacent to an opening of the left atrial appendage; advancing the distal end of the catheter through the opening and into the left atrial appendage; contacting a wall of the left atrial appendage with the deployable member and adhering the deployable member with the wall of the left atrial appendage; retracting the catheter proximally to move the wall from an original position to a second position, wherein in the second position, the left atrial appendage wall is at least partially inverted thereby reducing the internal volume of the left atrial appendage; and inserting adhesive into the left atrial appendage to secure the left atrial appendage in the second position.

Optionally, partially inverting the left atrial appendage wall comprises forming a peak with the left atrial appendage wall with an annular pocket.

Optionally, partially inverting the left atrial appendage wall comprises invaginating the left atrial appendage.

Optionally, the deployable member is a cryocooled member, the method further comprising cooling the crycooled member before adhering the crycooled member with the wall of the left atrial appendage.

Optionally, the crycooled member is a cryocooled tip of the catheter or a cryoballoon.

Optionally, when the crycooled member is a cryoballoon, the cryoballoon is collapsed when the catheter is positioned adjacent to the opening of the left atrial appendage and expanded before adhering the cryoballoon with the wall of the left atrial appendage.

Optionally, the method further comprises increasing an internal pressure of the cryoballoon before proximally retracting the left atrial appendage wall with the deployable member.

Optionally, the method further comprising incorporating radiopaque markers in the adhesive.

Optionally, the method further comprises curing the adhesive before the deployable member is unadhered from the left atrial appendage wall.

Optionally, inserting the adhesive comprises: providing a second steerable catheter; inserting a distal end of the second catheter towards the opening of the left atrial appendage; and delivering adhesive in the at least partially inverted portion of the left atrial appendage.

A further aspect of the invention provides a method of occluding a left atrial appendage, the method comprising: providing a catheter with an elongate tubular body and a central lumen that extends from a proximal end to a distal end of the catheter, the catheter comprising a deployable member arranged at the distal end of the catheter; positioning the catheter adjacent to an opening of the left atrial appendage; advancing the distal end of the catheter through the opening and into the left atrial appendage; puncturing a wall of the left atrial appendage with the deployable member to form an aperture and passing the deployable member through the aperture; expanding the deployable member to abut against an external surface of the left atrial appendage wall; retracting the catheter proximally to move the wall from an original position to a second position, wherein in the second position, the left atrial appendage wall is at least partially inverted thereby reducing the internal volume of the left atrial appendage; and inserting adhesive into the left atrial appendage to secure the left atrial appendage in the second position.

Optionally, the method further comprises collapsing the deployable member and proximally retracting the catheter to pass the deployable member through the aperture after inserting the adhesive into the left atrial appendage.

Optionally, the method further comprises closing the aperture after the adhesive is inserted into at least a portion of the left atrial appendage.

Optionally, partially inverting the left atrial appendage wall comprises forming a peak with the left atrial appendage wall with an annular pocket.

Optionally, partially inverting the left atrial appendage wall comprises invaginating the left atrial appendage.

Optionally, the deployable member is a cryocooled member, and after expanding the deployable member the deployable member is cryoadhered to the external surface of the left atrial appendage wall.

Optionally, the cryocooled member is a cryocooled tip of the catheter or a cryoballoon.

Optionally, when the cryocooled member is a cryoballoon, the cryoballoon is collapsed when positioned adjacent to the opening of the left atrial appendage.

Optionally, the method further comprises increasing an internal pressure of the cryoballoon before proximally retracting the left atrial appendage wall with the deployable member.

Optionally, the method further comprises incorporating radiopaque markers in the adhesive.

Optionally, the method further comprises curing the adhesive before collapsing the deployable member and passing the deployable member through the aperture.

Optionally, inserting the adhesive comprises: providing a second steerable catheter; inserting a distal end of the second catheter towards the opening of the left atrial appendage; and delivering adhesive in the at least partially inverted portion of the left atrial appendage.

A further aspect of the invention provides an occlusion device for occluding a left atrial appendage, the device comprising: a first catheter with a proximal end and a distal end, the distal end arranged to be positioned at an opening of the left atrial appendage; a central lumen extending from a proximal portion to a distal portion of the first catheter; a deployable member arranged at the distal end of the first catheter; a second catheter within the central lumen of the first catheter and arranged to deliver adhesive to the distal end of the first catheter.

Optionally, the device further comprises a pressure sensor for sensing the pressure in the deployable member at the distal end of the catheter, wherein the pressure sensor is arranged to send a signal to a control system if a measured pressure value crosses a predetermined threshold.

Optionally, the deployable member is a cryocooled member, and optionally, the cryocooled member is a cryocooled tip or a cryoballoon.

Optionally, the deployable member is a cryoballoon, only a portion of an external surface of the cryoballoon is arranged to be cooled, and preferably, wherein the portion of the external surface is at a distal end of the cryoballoon.

Optionally, the second catheter is a steerable delivery catheter and optionally, a distal end of the second catheter comprises a nozzle for accurately dispelling adhesive.

Optionally, the adhesive is a bioglue.

### BRIEF DESCRIPTION OF THE DRAWINGS

Embodiments of the invention will now be described with reference to the accompanying drawings, in which:

FIG. 1 is a schematic sectional illustration of a heart;

FIG. 2 illustrates a diagrammatic view of a patient undergoing a cardiovascular procedure to occlude a left atrial appendage;

FIG. 3 illustrates a schematic sectional illustration of an exemplary left atrial appendage;

FIGS. 4a and 4b illustrate an exemplary catheter;

FIGS. 5a-5d are schematic views of an occlusion device being delivered to a left atrial appendage;

FIG. 6 illustrates a flowchart showing an exemplary method of occluding a left atrial appendage;

FIG. 7 illustrates a catheter adjacent to a left atrial appendage;

FIG. 8 illustrates a distal end of a catheter inserting into the left atrial appendage;

FIG. 9 illustrates the catheter contacting and adhering the left atrial appendage wall;

FIG. 10 illustrates the left atrial appendage in a partially inverted position;

FIG. 11 illustrates a flowchart showing an exemplary method of occluding a left atrial appendage;

FIG. 12 illustrates a distal end of the catheter puncturing the left atrial appendage wall according to a further example of the invention;

FIG. 13 illustrates the left atrial appendage in a partially inverted position according to a further example of the invention;

FIGS. 14a and 14b illustrates a cross-sectional view of the catheter.

### DETAILED DESCRIPTION OF EMBODIMENT(S)

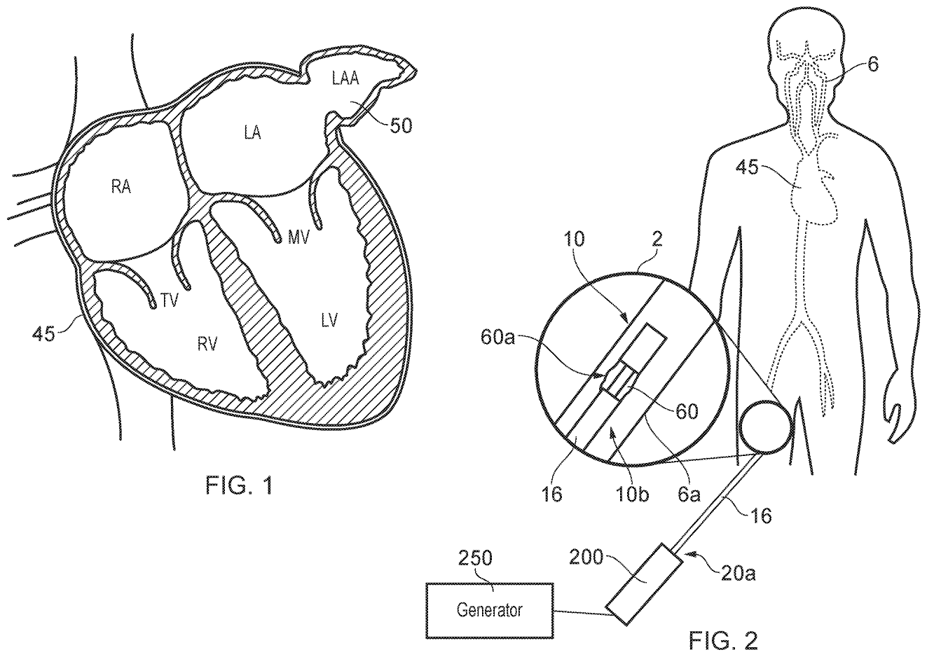

FIG. 1 is a schematic sectional illustration of a human heart 45 that depicts the four heart chambers (right atrium RA, right ventricle RV, left atrium LA, left ventricle LV) and a left atrial appendage LAA 50.

FIG. 2 illustrates a diagrammatic view of a patient undergoing an exemplary left atrial appendage occlusion procedure. As indicated by the enlarged circle area 2, an occlusion device 10 may be inserted into an artery 6a (e.g. a femoral artery). The occlusion device 10 includes a catheter 20 that is arranged to be positioned next to the left atrial appendage 50 within the heart 45. The occlusion device 10 is formed of a proximal portion (not shown) and a distal portion 10b. The proximal portion of the occlusion device 10 will remain outside of the patient's body while the distal portion 10b is advanced through the patients' vasculature 6 towards the left atrial appendage 50.

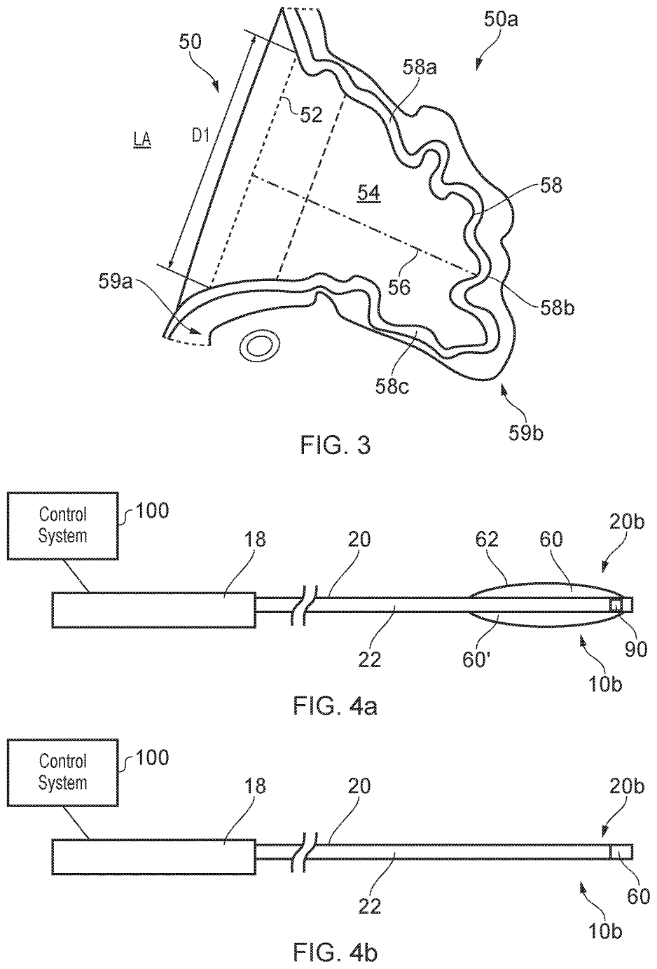

The occlusion device 10 is arranged to occlude (or obstruct) a left atrial appendage 50. As shown in FIG. 3, the left atrial appendage 50 is generally bursiform (pouch) shaped and has an opening 52 at a proximal end 59a that extends to a distal end 59b. The opening 52 is generally circular in shape and has a diameter D1. As shown, the opening 52 generally corresponds to the widest part of the left atrial appendage 50.

The left atrial appendage 50 has an internal volume 54 that is in fluid communication with the left atrium LA. The internal volume 54 may contain a volume of blood from the heart 45. The internal volume 54 is partially enclosed by a wall 58. As shown, the wall 58 has an irregular shape that generally forms an ear-shaped structure. In this example, the left atrial appendage wall 58 is formed of a superior wall 58a, an inferior wall 58c and a rear wall 58b.

The left atrial appendage 50 may have any type of morphology. For example, the left atrial appendage 50 may have a depth greater than the opening diameter D1, a depth smaller than the opening diameter D1, have a chicken-wing shape, cactus shape or cauliflower shape.

The left atrial appendage has a depth 56 that extends from the opening 52 (at the proximal end 59a) to the rear wall 58b (at the distal end 59b of the LAA). Generally, the depth 56 of the left atrial appendage 50 is greatest at the rear wall 58b. The left atrial appendage 50 is in a natural pre-treatment state 50a in FIG. 2.

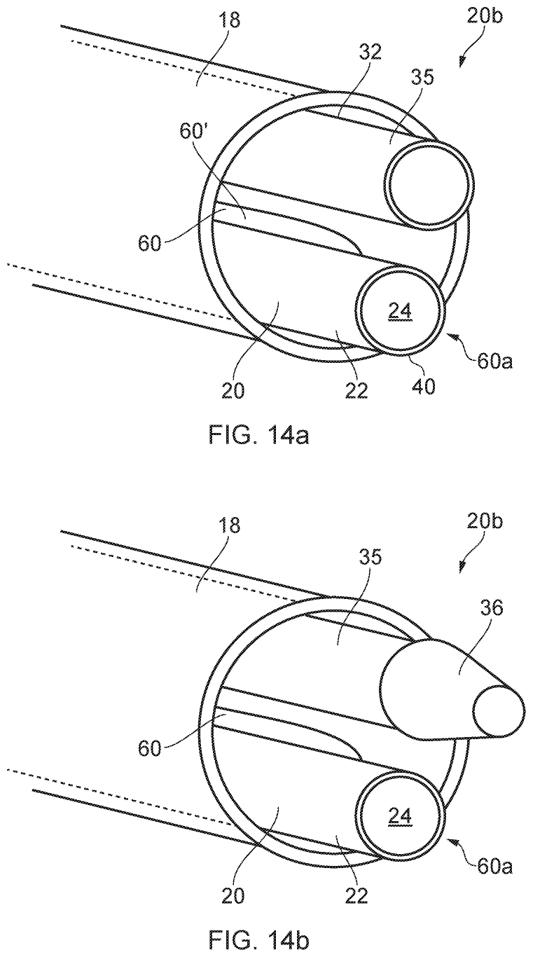

Two exemplary distal catheter portions 20bs are shown in FIGS. 4a and 4b. As shown, the catheter 20 is housed in a delivery catheter 18 and is electronically connected to a control system 100. The catheter 20 has an elongate tubular body 22 that extends from the proximal portion (not shown) to the distal portion 20b of the catheter 20. The catheter 20 has a central lumen 24 (shown more clearly in FIG. 14b) that extends from a proximal end 20a of the catheter 20 to the distal end 20b of the catheter 20. The central lumen 24 extends to an aperture 40 at the distal end 20b of the tubular body 22.

As shown schematically in FIG. 2, the proximal end 20a of the catheter 20 and the delivery catheter 18 are housed in a handle 200 of the occlusion device 10. The proximal end 20a of the catheter therefore remains outside of the patient's body. A medical practitioner manipulates the position and orientation of the catheter 20 by using the handle 200. As shown, the handle 200 may also be connected to any number of systems known to those skilled in the art, such as a generator 250.

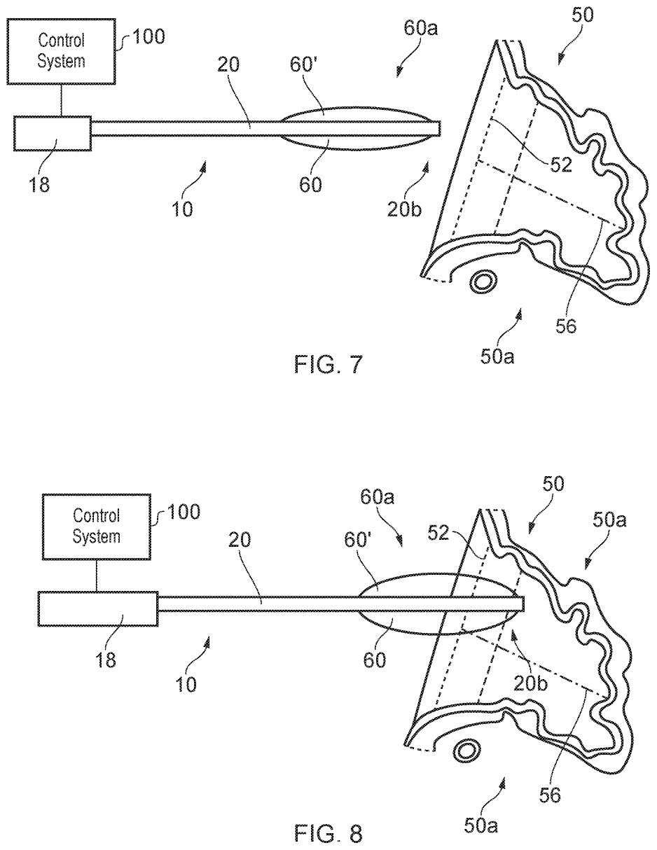

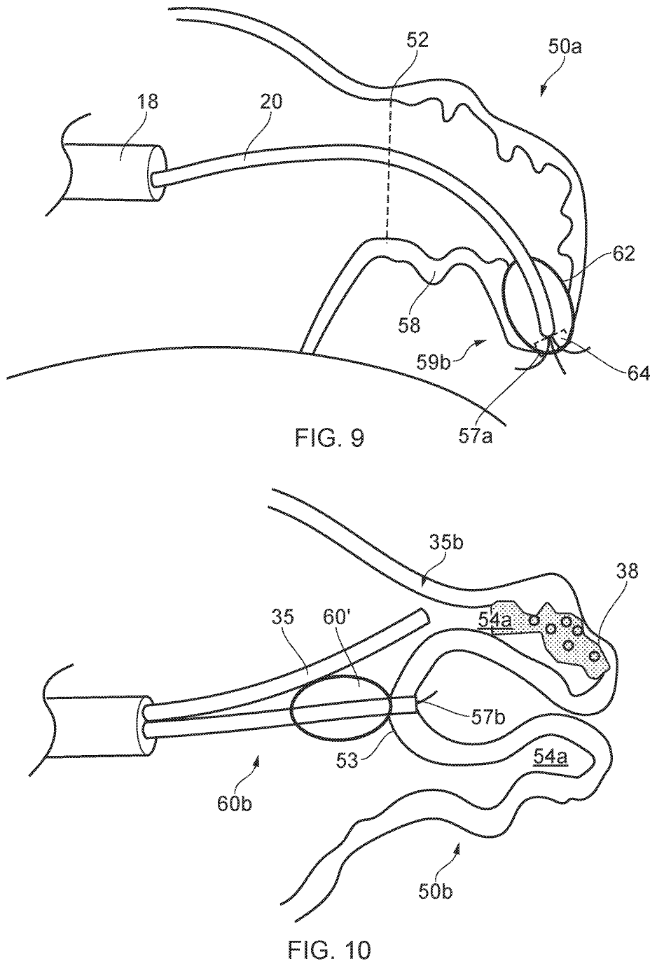

A deployable member 60 is arranged at the distal end 20b of the catheter 20. The deployable member 60 may be coated, for example with a special coating or material doping, to ensure greater visibility under fluoroscopy and echography. The deployable member 60 is arranged to contact and adhere to the left atrial appendage wall 58 at the distal end 59b of the LAA. The deployable member 60 may be an expandable balloon 60′ (as shown in FIG. 4a) that is arranged to move from a collapsed state 60a to an inflated state 60b (shown in FIG. 9). In this example, the balloon 60′ is in a collapsed state 60a (i.e. not inflated) when the catheter 20 is moved through the patient's vasculature 6 towards the left atrial appendage 50. As shown in FIG. 7, the deployable member 60 remains in a collapsed state 60a when the catheter 20 is positioned near the opening 52 of the left atrial appendage 50.

Preferably, the deployable member 60 is a cryocooled member, such as a deployable cryoballoon. When the cryoballoon is in an inflated state 60b, the balloon 60′ contains cryogenic fluid (cryo-fluid) or refrigerant. An outer surface 62 of the cryoballoon 60′ contacts the left atrial appendage wall 58 to cryoadhere to the wall. The outer surface 62 of the cryoballoon may have a temperature between 0 and −15° C. This ensures that the outer surface 62 may cryoadhere to the wall 58. However, the outer surface 62 of the cryoballoon may be at any suitable temperature to achieve cryoadhesion between the cryoballoon and the tissue 58.

Preferably, only a portion 64 of the outer surface 62 of the balloon 60′ cryoadheres to the wall. The portion 64 is, in one example, formed from a different material (such as polytetrafluoroethylene (PTFE)) from the rest of the outer surface 62, so that only the external surface of the portion 64 becomes cooled when the balloon is in an inflated state 60b. The portion 64 may be any suitable shape and size to successfully adhere to the wall 58 when the balloon is in an inflated state 60b, e.g., filled with cryogenic fluid/refrigerant. As shown more clearly in FIG. 9, the portion 64 is preferably at a distal end of the deployable member 60. This prevents the deployable member 60 from adhering to the left atrial appendage wall 58 as the catheter 20 advances into the left atrial appendage 50.

In other examples, as shown in FIG. 4b, the deployable member 60 may be a cryocooled tip 60. The cryocooled tip 60 is at the distal end 20b of the tubular body 22 of the catheter 20 and may be formed in or onto the tubular body 22 of the catheter 20. The cryocooled tip 60 is also cooled using cryogenic fluid or refrigerant. In this example, the cryocooled tip 60 does not move between a deployed and undeployed state but may be selectively cooled with cryogenic fluid/refrigerant after the tip 60 contacts the left atrial appendage wall 58.

The deployable member 60 may be any suitable member at the distal end 20b of the catheter 20. The deployable member 60 may adhere to the wall 58 of the left atrial appendage 50 through any suitable means other than cryoadherance, for example, through adhesive (such as bio-glue) or through mechanically engaging with the wall 58.

When the deployable member 60 is a balloon or cryoballoon, the catheter 20 may also include a pressure sensor 90, shown schematically in FIG. 4a. The pressure sensor 90 is electronically connected to the control system 100. The pressure sensor 90 may be positioned at any suitable position at the distal end 20b of the balloon catheter 20, but preferably is housed within the deployable member 60.

The pressure sensor 90 is arranged to measure the pressure inside the deployable member 60. The pressure inside the deployable member 60 is observed by the medical practitioner to ensure that the deployable member 60 is in a correct configuration (i.e. collapsed state 60a or inflated state 60b) before the device 60 is inserted into the left atrial appendage 50. The internal pressure of the device 60 may also be increased before the catheter 20 is retracted proximally to displace the atrial wall 58 (discussed further below).

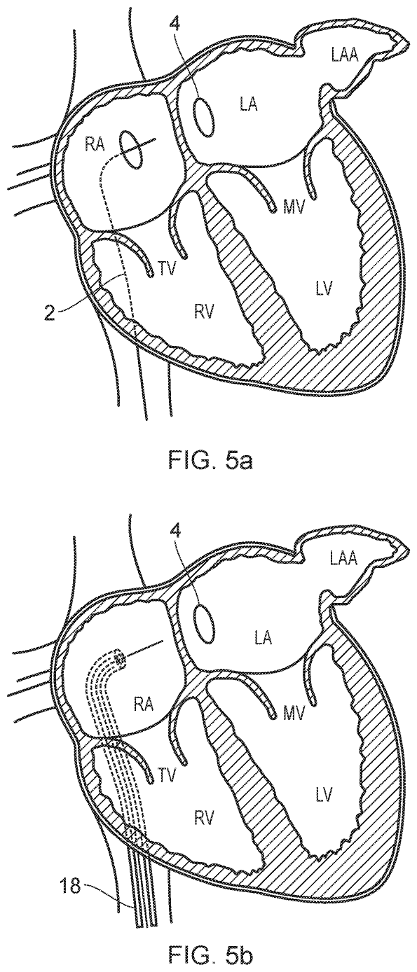

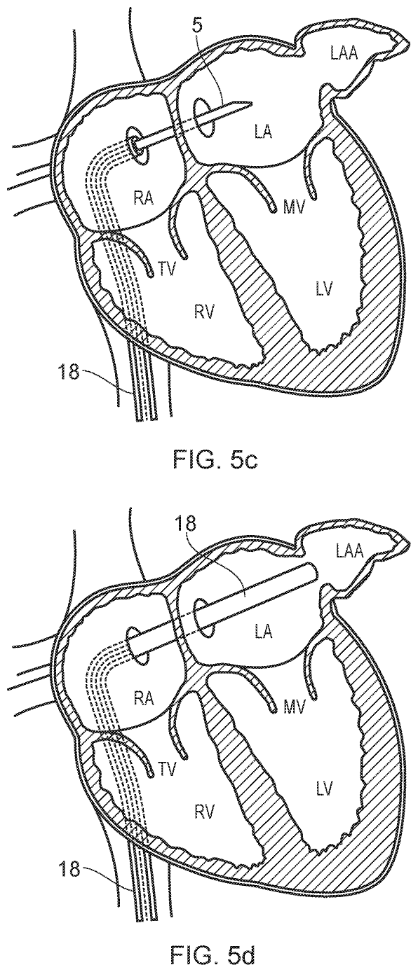

Referring to FIGS. 5A to 5D, a method for delivering and deploying the occlusion device 10 to a left atrial appendage will now be described in more detail. In an exemplary embodiment, a guidewire 2 is advanced after having been introduced into the vasculature 6 via a percutaneous entry point and tracked through the vasculature into a left atrium LA of a heart 45. Intravascular access to the right atrium RA may be achieved via a percutaneous access site to femoral venous access up to the inferior vena cava, or other known access routes. Thereafter, a guidewire is advanced through the circulatory system, eventually arriving at the heart.

The guidewire 2 is directed into the right atrium RA (see FIG. 5A), traverses the right atrium and is made to traverse, with the aid of a pre-existing hole 4 (see FIG. 5B) or a transseptal needle 5 (see FIG. 5C) an atrial septum, thereby entering the left atrium LA. Once the guidewire is positioned, the entry port and the atrial septum are dilated to permit entry of the delivery catheter 18 into the left atrium LA towards the left atrial appendage LAA 50 (see FIG. 5D).

The distal end 20b of the catheter 20 is positioned proximate the left atrial appendage LAA 50, as shown in FIG. 7. Although described as a transfemoral antegrade approach for percutaneously accessing the left atrium LA, the catheter 18 may be positioned within the desired area of the heart via different methods or routes. For example, and not by way of limitation, another possible path would be through the radial vein into the brachial vein, through the subclavian vein, through the superior vena cava into the right atrium, and then transeptally into the left atrium. In another example, another path may be from the femoral vein up through the inferior vena cava and into the right atrium, and transeptally into the left atrium.

Yet another possible path would be through the femoral artery into the aorta (as shown in FIG. 2), through the aortic valve into the left ventricle, and then retrograde through the mitral valve into the left atrium. In another embodiment, the left ventricle LV may be accessed via a transapical approach, and the catheter 18 may be advanced through the left ventricle LV, the mitral valve, and into the left atrium LA adjacent the left atrial appendage LAA. In addition, although described with the use of a guidewire, in another embodiment hereof the delivery catheter 18 may access the left atrium LA without the use of a guidewire.

The distal end 20b of the catheter 20 is advanced into the left atrial appendage 50 so that the deployable member 60 contacts the atrial wall 58. As shown in FIG. 9, the deployable member 60 contacts the atrial wall 58 at the distal end 59b of the left atrial appendage 50. In some instances, it may be preferable to contact the distal end 59b of the left atrial appendage 50 so that when the catheter 20 is retracted and the left atrial appendage is partially inverted, the reduced internal volume is relatively equally distributed. However, the deployable member 60 may contact any wall 58a-58c of the left atrial appendage.

If the deployable member 60 is a cryocooled member, it is cooled with cryogenic fluid or refrigerant at this stage so that the deployable member 60 cryoadheres to the left atrial appendage wall 58 at the point of contact. The deployable member 60 is therefore securely attached to the atrial wall 58 at this stage.

If the deployable member 60 is a balloon 60′, the pressure of the deployable member 60 may be increased after the deployable member 60 is adhered with the wall 58. The pressure of the deployable member 60 may be increased by inflating the balloon 60′ with more fluid or gas. Increasing the pressure of the deployable member 60 improves the rigidity of the deployable member 60 so the deployable member 60 is not as flexible. This enables the medical practitioner to exert less force when retracting the catheter 20 proximally to displace the left atrial appendage wall 58. The deployable member 60 may be secured to the catheter 20 through a strengthened connector (not shown) so that the deployable member 60 can be retracted without breaking away from the catheter 20.

The pressure sensor 90 is electrically connected to a control system 100 and communicates the measured pressure reading to the control system 100. The pressure sensor 90 may continuously measure the pressure inside the deployable member 60 as the left atrial appendage is partially inverted or may only measure the internal pressure after the pressure has been increased. The measured pressure is communicated to the control system 100 which establishes whether or not the measured pressure crosses a predetermined threshold, T. When the deployable member 60′ is a balloon 60′, the predetermined threshold T may be indicative of the balloon 60′ becoming stiffer. Typically, the balloon 60′ is formed from a deformable material, so that the outer surface 62 of the balloon 60′ easily deforms and adjusts to the shape of the left atrial appendage wall 58. This enables the balloon 60′ to have a larger area to contact against the left atrial appendage wall 58. If the balloon 60′ is over pressurised, the outer surface 62 of the balloon 60′ is less deformable. Less area of the outer surface 62 of the balloon 60′ therefore contacts against the left atrial appendage wall 58.

In other examples, when the deployable member 60 is a balloon 60′, the predetermined threshold T may be indicative of the size of the deployable member 60. If the pressure reaches the threshold T, it may indicate that the balloon 60′ is too large. If the balloon 60′ is too large, then it may impact the ability of the medical practitioner to proximally retract the balloon 60 to invaginate the left atrial appendage 50, e.g. if the balloon 60′ is larger than the opening 52 of the left atrial appendage.

If the measured pressure does cross the predetermined threshold, T, the control system 100 is arranged to emit an alarm to alert the medical practitioner. The medical practitioner may then stop inflating the deployable member 60 further, or they may reduce the pressure inside the deployable member 60 by partially deflating the deployable member 60.

If the measured pressure does not exceed a predetermined threshold T after adhering to the wall, the medical practitioner then proceeds to proximally retract the catheter 20 to partially invert the left atrial appendage 50 to an inverted state 50b. If the deployable member 60 is not a balloon, or if the medical practitioner does not want to increase the internal pressure of the deployable member, then the medical practitioner retracts the catheter proximally.

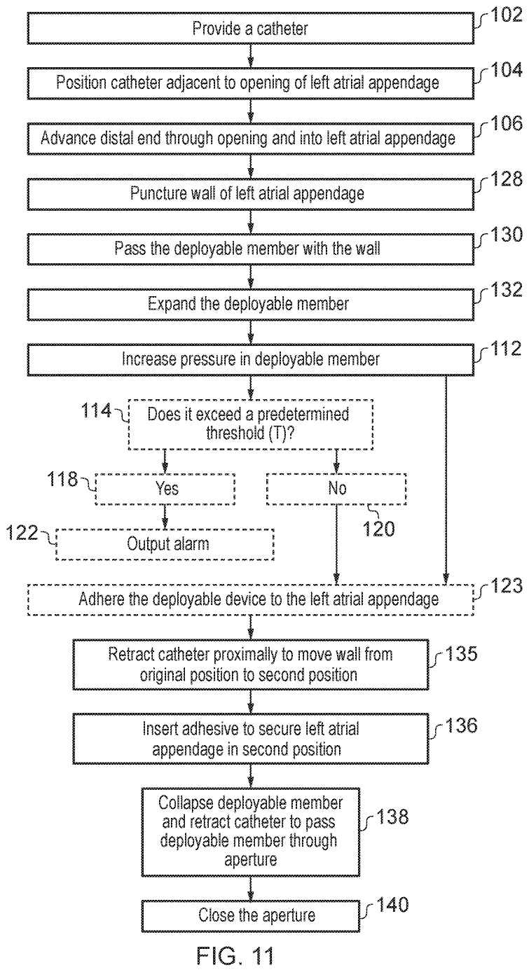

As shown in FIG. 9, the left atrial appendage 50 is in a natural pre-treatment state 50a. In this position, the wall 58 at the distal end 59b of the LAA 50 is in a first position 57a, which generally corresponds to the natural morphology of the left atrial appendage 50. After the deployable member 60 has adhered to the wall 58, the medical practitioner retracts the catheter 20 proximally. Proximally retracting the catheter 20 moves the wall 58 from the first position 57a to a second position 57b, as shown in FIGS. 10 and 11. As shown, partially inverting the left atrial appendage includes invaginating the left atrial appendage 50. The wall 58 of the left atrial appendage is folded back in towards the left atrium LA to form a reduced volume 54a.

The catheter 20 may include a force gauge (not shown). The force gauge may be electrically connected to the control system 100 and communicates the measured force exerted by the catheter 20 when the catheter 20 is retracted proximally by the medical practitioner. The force gauge may measure the force as the left atrial appendage is inverted. The measured force is communicated to the control system 100, which establishes whether or not the measured force crosses a predetermined threshold. The predetermined threshold may be used to determine how far the left atrial appendage wall 58 has been inverted, or that that the left atrial appendage wall 58 has been fully inverted.

The control system 100 may indicate to the medical practitioner how much force to apply to the catheter 20 based on the relative position of the left atrial appendage 50. The control system 100 may determine the position of the left atrial appendage 50 based on the reading from the pressure sensor 90, or through other means, such as imaging. Optionally, the deployable member may include electrodes (not shown) on the outer surface 62 of the deployable member 60. The electrodes may be used to monitor the contact and the cryoadhered surface area between the tissue and the deployable member 60. The control system 100 may use the measurements obtained from the electrodes to determine how much force should be applied to the catheter 20. In this arrangement, the control system 100 helps prevent the medical practitioner from causing mechanical trauma on the left atrial appendage 50 by exerting excessive force as the catheter 20 is retracted proximally.

FIG. 10 is a cross-sectional schematic side view of a left atrial appendage 50 in an inverted state 50b. In the inverted state 50b, the left atrial appendage wall 58 is in a second position 57b, where the appendage wall 58 is at least partially inverted. In the second position 57b, the left atrial appendage wall 58 is proximal to the left atrium LA compared to the first position 57a in FIG. 10.

In the second position 57b, the left atrial appendage wall 58 forms a peak 53. As shown, the peak 53 is surrounded by a reduced volume 54a. In FIG. 10, the reduced volume 54a is shown as two pockets that are superior and inferior to the peak 53, however, the reduced volume 54a extends around the peak 53. The reduced volume 54a is less than the original internal volume 54 of the left atrial appendage 50. The reduced volume 54a is a pocket that surrounds the peak 53.

Adhesive 30 is inserted into the left atrial appendage 50 to secure the left atrial appendage in the partially inverted state 50b. As shown in FIG. 10, the adhesive 30 is delivered into the reduced volume 54a. The adhesive 30 contacts the surrounding atrial appendage walls 58 to secure the walls together. The adhesive 30 is preferably delivered to the left atrial appendage 50 through a second steerable catheter 35. The second catheter 35 has a proximal end (not shown) and a distal end 35b. The second catheter 35 has a tubular body 32 that extends to an aperture 40 as shown in FIG. 14a.

The second catheter 35 is arranged to deliver adhesive 30 through the aperture 40. Preferably, the distal end 35b of the second catheter 35 includes a nozzle 36 that is used to accurately dispel adhesive 30 into the left atrial appendage 50. The nozzle 36 may be any suitable shape or size to accurately dispel the preferred volume of adhesive 30 into the left atrial appendage 50.

As shown in FIGS. 10, 13, 14a and 14b, the second catheter 35 may be introduced through the lumen 24 of the catheter 20 or alternatively, the second catheter 35 may be provided outside of the catheter 20. In both examples, the distal end 35b of the balloon catheter 30 is arranged to deliver adhesive to the reduced volume 54a of the left atrial appendage 50.

The adhesive 30 may be any suitable bioglue, such as cyanoacrylate-based adhesive, bovine serum albumin-glutaraldehyde-based, hydrogel based or animal-inspired adhesive. Preferably, the bioglue is any suitable medically inert substance that is arranged to adhere to tissue, such as the LAA walls. The adhesive 30 may be incorporated with radiopaque markers 38 (shown schematically in FIG. 13) so that a medical practitioner may observe the bioglue using relevant imaging techniques, such as fluoroscopy.

In one example, the adhesive 30 is inserted and cured before the deployable member 60 is unadhered from the left atrial appendage wall 58. The adhesive 30 may be arranged to cure after a period of time, or by using a curing agent, such as a light source (not shown). The light source may, for example, be an ultraviolet (UV) light source or visible light source. Once the adhesive 30 is cured, if the deployable member 60 is a balloon 60′, the balloon 60′ is returned to a collapsed state 60a before withdrawing the catheter 20 from the heart 45 and leaving the left atrial appendage 50 secured in the partially inverted state 50b.

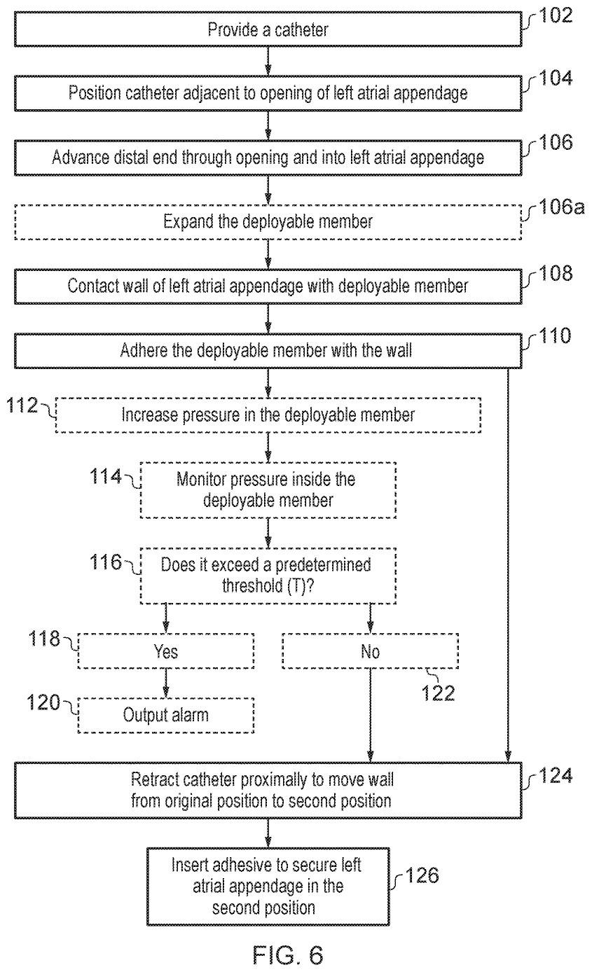

An exemplary occlusion device 10 for occluding a left atrial appendage 50 is described above. An exemplary method for occluding the left atrial appendage 50 is described below in relation to the flow chart shown in FIG. 6.

At step 102, a catheter 20 is provided. Once the distal end 20b of the catheter 20 is adjacent to the opening at step 104, the distal end 20b of the catheter 20 is advanced through the opening 52 towards the distal end 59b of the left atrial appendage 50. As shown in FIG. 8, the distal end 20b of the catheter is positioned into the volume 54 of the left atrial appendage 50 at step 106.

If the deployable member 60 is a balloon 60′, the deployable member 60 is preferably expanded (i.e. in the inflated state 60b) at step 106 before the deployable member 60 contacts the atrial wall 58 at step 108. The balloon 60′ may be inflated, for example, with gas or fluid.

At step 108, the deployable member 60 is moved to contact a wall 58a-c of the left atrial appendage 50. Preferably, a large surface area of the deployable member 60 contacts the left atrial wall 58 so that a large surface area adheres to the wall 58 at step 110. A larger surface area improves the strength of the connection between the deployable member 60 and the wall 58 when the catheter 20 is retracted proximally in step 124.

At step 110, the deployable member 60 is adhered to the wall 58 at the point of contact. This may be by using adhesive, but preferably the deployable member 60 is a cryocooled member so that the deployable member 60 cryoadheres to the left atrial appendage wall.

As described earlier, the occlusion device 10 may include a pressure sensor 90. The pressure sensor 90 may continuously measure the pressure inside the deployable member 60 as the left atrial appendage is partially inverted or may only measure the internal pressure of the deployable member 60 after the pressure has been increased in step 112.

If the internal pressure of the deployable member 60 has been increased at step 112, the medical practitioner or the control system 100 may monitor the pressure of the deployable member 60. If the measured pressure does exceed the predetermined threshold, T at step 115, the control system 100 emits an alarm at step 120 to alert the medical practitioner. The medical practitioner may then stop inflating the deployable member 60 further, or they may reduce the pressure inside the deployable member 60 by partially deflating the deployable member 60.

If the measured pressure does not exceed a predetermined threshold T after adhering to the wall 58 at step 122, the medical practitioner then proceeds to step 124 to proximally retract the catheter 20 to partially invert the left atrial appendage 50 to an inverted state 50b. Furthermore, if the deployable member 60 is not a balloon, or if the medical practitioner does not want to increase the internal pressure of the deployable member, then the medical practitioner moves directly onto step 124 to retract the catheter proximally.

Retracting the catheter 20 after the deployable member 60 has been adhered to the wall moves the wall of the left atrial appendage 50 from a first position 57a to a second position 57b, as described earlier. The first position 57a of the left atrial appendage wall generally corresponds to the position of the wall when the left atrial appendage is in a natural, pre-treatment state 50a. The second position 57b of the left atrial appendage wall corresponds to the position of the wall when the left atrial appendage is in an inverted state 50b.

At step 126, adhesive is delivered into the left atrial appendage 50 to secure the left atrial appendage wall in the second position 57b, thereby securing the left atrial appendage 50 in the inverted state 50b. As described earlier, preferably, the adhesive 30 is delivered to the left atrial appendage 50 through the second steerable catheter 30 but may be delivered to the left atrial appendage 50 through any suitable means. Once the adhesive 30 has cured, the deployable member 60 is removed from the left atrial appendage wall 58 and the occlusion device 10 is removed from the patient.

Another method of partially inverting the left atrial appendage is shown in FIGS. 11-13. In this example, the catheter 20 is positioned and advanced into the left atrial appendage 50 in identical steps 102-106 as described above. The deployable member 60 is preferably a balloon 60′, however any suitable expanding member may be used. The balloon 60′ is kept in a collapsed state 60a as the distal end 20b of the catheter 20 is inserted into the left atrial appendage 50. The distal end 20b of the catheter 20 is inserted towards the distal end 59b of the left atrial appendage 50.

At step 128, the distal end 20b of the catheter 20 punctures the left atrial appendage wall 58 to form an aperture 70. 60 In one example, the distal end 20b may puncture the left atrial appendage wall 58 using a needle, or by using a RF needle. These may be integrated into the distal end 20b of the catheter 20 or may be inserted through the inner lumen 24 of the catheter 20. In other examples, the distal end of the deployable member 60 may be used to form the aperture 70. Alternatively, the aperture 70 may be formed by another device such as mechanism with multiple tines. The mechanism (not shown) may also act as an anchor to the deployable member 60, ensuring that the aperture 70 does not tear further when the left atrial appendage 50 is inverted.

Once the aperture 70 is formed, the medical practitioner distally pushes the catheter 20 so that the deployable member 60 is passed through the aperture 70 at step 130. The deployable member 60 is in an undeployed state 60a as the member is passed through the aperture so that the size of the aperture 70 is minimised. The deployable member 60 is passed through the aperture 70 so that it is positioned outside the heart 45 and outside the left atrial appendage 50.

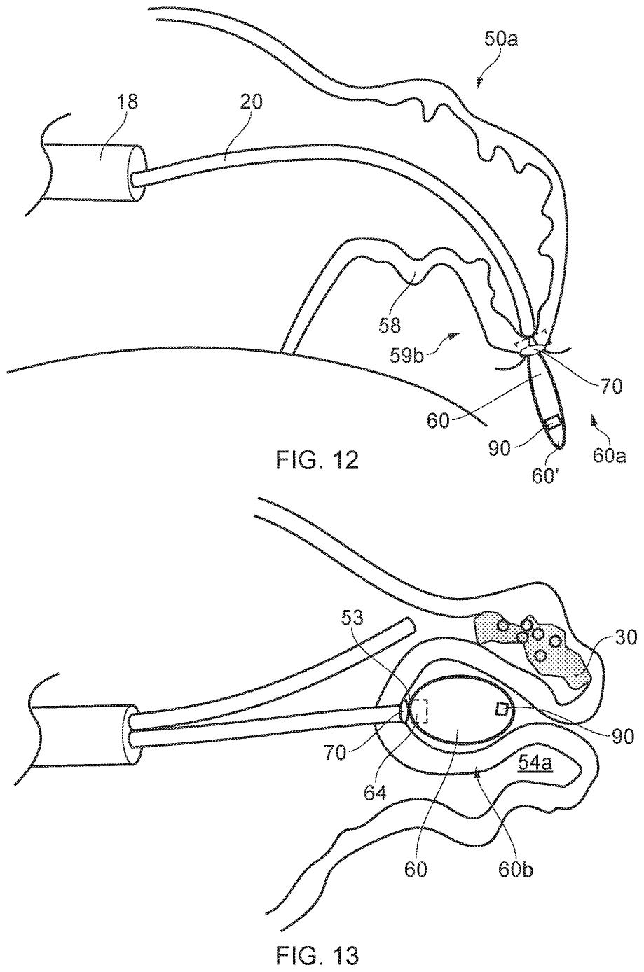

The deployable member 60 is then expanded into an inflated state 60b. In this example, a proximal portion of the outside surface 62 of the deployable member contacts against an external surface of the left atrial appendage 50. As described earlier in relation to FIG. 6, the medical practitioner may increase the internal pressure of the deployable member 60 at step 112. However, it will be understood that the pressure sensor in FIGS. 12 and 13 operates in an identical manner to the sensor 90 shown in FIG. 9 and described in FIG. 6.

The deployable member 60 may also be cryoadhered to the outside of the left atrial appendage 50 in step 123. As shown in FIG. 13, the portion 64 of the outer surface 62 of the deployable member 60 is located at the proximal end of the device. This ensures that the area of the deployable member 60 that contacts the left atrial appendage 60 can adhere to the left atrial appendage.

At step 135, the catheter 20 is retracted proximally to move the wall 58 from an original position 57a to a second position 57b. As shown, the catheter 20 is retracted to form a peak 53 similar to the inverted state 50b shown in FIGS. 9 and 10. In this example, the deployable member 60 provides a force on the external surface of the appendage wall 58 to form the peak 53. The left atrial appendage 50 is secured in the inverted state 50b by delivering adhesive 30 into the reduced volume 54a.

Once the adhesive 30 has cured, the deployable member 60 is returned to a collapsed state 60a before being passed through the aperture 70 by proximally retracting the catheter 20. The aperture 70 may be adhered closed by using additional adhesive or biofoam (for example, from the steerable catheter 30), or may be left to naturally heal. In other examples, a detachable plug may be positioned into the aperture 70 using tines or barbs. The detachable plug may have an opening that allows the catheter 20 with the deployable member 60 to pass through the plug. The plug may be positioned in the aperture 70 before the deployable member 60 is passed through the plug and the aperture 70. Once the left atrial appendage has been retracted and the deployable member 60 is passed back through the aperture 70, the detachable plug may be arranged to seal itself back through integral haemostatic valve (e.g. using polymer membrane slits).

With regard to the terms “distal” and “proximal” within this description, unless otherwise specified, the terms can reference a relative position of the portions of the delivery catheter system with reference to a medical practitioner and/or a location in the vasculature or heart. For example, “proximal” can refer to a position closer to the medical practitioner of the device or an incision into the vasculature, and “distal” can refer to a position that is more distant from the medical practitioner of the device or further from the incision along the vasculature (e.g., the end of the catheter).

Where the word ‘or’ appears this is to be construed to mean ‘and/or’ such that items referred to are not necessarily mutually exclusive and may be used in any appropriate combination. Although the teachings have been described above with reference to one or more preferred embodiments, it will be appreciated that various changes or modifications may be made without departing from the scope as defined in the appended claims.

### Clauses

Clause 1. A method of occluding a left atrial appendage, the method comprising: providing a catheter with an elongate tubular body and a central lumen that extends from a proximal end to a distal end of the catheter, the catheter comprising a deployable member arranged at the distal end of the catheter;
positioning the catheter adjacent to an opening of the left atrial appendage;
advancing the distal end of the catheter through the opening and into the left atrial appendage;
contacting a wall of the left atrial appendage with the deployable member and adhering the deployable member with the wall of the left atrial appendage; and
retracting the catheter proximally to move the wall from an original position to a second position, wherein in the second position, the left atrial appendage wall is at least partially inverted thereby reducing the internal volume of the left atrial appendage; and
inserting adhesive into the left atrial appendage to secure the left atrial appendage in the second position.

Clause 2. The method of occluding the left atrial appendage according to clause 1, wherein partially inverting the left atrial appendage wall comprises forming a peak with the left atrial appendage wall with an annular pocket.

Clause 3. The method of occluding the left atrial appendage according to clause 1 or 2, wherein partially inverting the left atrial appendage wall comprises invaginating the left atrial appendage.

Clause 4. The method of occluding the left atrial appendage according to any preceding clause, wherein the deployable member is a cryocooled member, the method further comprising cooling the cryocooled member before adhering the cryocooled member with the wall of the left atrial appendage.

Clause 5. The method of occluding the left atrial appendage according to clause 4, wherein the cryocooled member is a cryocooled tip of the catheter or a cryoballoon.

Clause 6. The method of occluding the left atrial appendage according to clause 5, wherein when the cryocooled member is a cryoballoon, the cryoballoon is collapsed when the catheter is positioned adjacent to the opening of the left atrial appendage and expanded before adhering the cryoballoon with the wall of the left atrial appendage.

Clause 7. The method of occluding the left atrial appendage according clause 6, the method further comprising increasing an internal pressure of the cryoballoon before proximally retracting the left atrial appendage wall with the deployable member.

Clause 8. The method of occluding the left atrial appendage according to any preceding clause, the method further comprising incorporating radiopaque markers in the adhesive.

Clause 9. The method of occluding the left atrial appendage according to any preceding clause wherein the method further comprises curing the adhesive before the deployable member is unadhered from the left atrial appendage wall.

Clause 10. The method of occluding the left atrial appendage according to any preceding clause, wherein inserting the adhesive comprises:
providing a second steerable catheter;
inserting a distal end of the second catheter towards the opening of the left atrial appendage; and
delivering adhesive in the at least partially inverted portion of the left atrial appendage.

Clause 11. A method of occluding a left atrial appendage, the method comprising:
providing a catheter with an elongate tubular body and a central lumen that extends from a proximal end to a distal end of the catheter, the catheter comprising a deployable member arranged at the distal end of the catheter;
positioning the catheter adjacent to an opening of the left atrial appendage;
advancing the distal end of the catheter through the opening and into the left atrial appendage;
puncturing a wall of the left atrial appendage with the deployable member to form an aperture and passing the deployable member through the aperture;
expanding the deployable member to abut against an external surface of the left atrial appendage wall;
retracting the catheter proximally to move the wall from an original position to a second position, wherein in the second position, the left atrial appendage wall is at least partially inverted thereby reducing the internal volume of the left atrial appendage; and
delivering adhesive into the left atrial appendage to secure the left atrial appendage in the second position.

Clause 12. The method of occluding the left atrial appendage according to clause 11, wherein the method further comprises collapsing the deployable member and proximally retracting the catheter to pass the deployable member through the aperture after inserting the adhesive into the left atrial appendage.

Clause 13. The method of occluding the left atrial appendage according to clause 11 or 12, wherein the method further comprises closing the aperture after the adhesive is inserted into at least a portion of the left atrial appendage.

Clause 14. The method of occluding the left atrial appendage according to any one of clauses 11 to 13, wherein partially inverting the left atrial appendage wall comprises forming a peak with the left atrial appendage wall with an annular pocket.

Clause 15. The method of occluding the left atrial appendage according to any one of clauses 11 to 14, wherein partially inverting the left atrial appendage wall comprises invaginating the left atrial appendage.

Clause 16. The method of occluding the left atrial appendage according to any one of clauses 11 to 15, wherein the deployable member is a cryocooled member, and after expanding the deployable member the deployable member is cryoadhered to the external surface of the left atrial appendage wall.

Clause 17. The method of occluding the left atrial appendage according to clause 16, wherein the cryocooled member is a cryocooled tip of the catheter or a cryoballoon.

Clause 18. The method of occluding the left atrial appendage according to clause 17, wherein when the cryocooled member is a cryoballoon, the cryoballoon is collapsed when positioned adjacent to the opening of the left atrial appendage.

Clause 19. The method of occluding the left atrial appendage according to clause 18, the method further comprising increasing an internal pressure of the cryoballoon before proximally retracting the left atrial appendage wall with the deployable member.

Clause 20. The method of occluding the left atrial appendage according to any one of clauses 11 to 19, the method further comprising incorporating radiopaque markers in the adhesive.

Clause 21. The method of occluding the left atrial appendage according to any one of clauses 12 to 20, wherein the method further comprises curing the adhesive before collapsing the deployable member and passing the deployable member through the aperture.

Clause 22. The method of occluding the left atrial appendage according to any one of clauses 11 to 21, wherein delivering the adhesive comprises:
providing a second steerable catheter;
inserting a distal end of the second catheter towards the opening of the left atrial appendage; and
delivering adhesive in the at least partially inverted portion of the left atrial appendage.

Clause 23. An occlusion device for occluding a left atrial appendage, the device comprising:
a first catheter with a proximal end and a distal end, the distal end arranged to be positioned at an opening of the left atrial appendage;
a central lumen extending from a proximal portion to a distal portion of the first catheter;
a deployable member arranged at the distal end of the first catheter;
a second catheter within the central lumen of the first catheter and arranged to deliver adhesive to the distal end of the first catheter.

Clause 24. The occlusion device according to clause 23, further comprising a pressure sensor for sensing the pressure in the deployable member at the distal end of the catheter, wherein the pressure sensor is arranged to send a signal to a control system if a measured pressure value crosses a predetermined threshold.

Clause 25. The occlusion device according to clause 23 or 24, wherein the deployable member is a cryocooled member, and optionally, the cryocooled member is a cryocooled tip or a cryoballoon.

Clause 26. The occlusion device according to clause 25, wherein when the deployable member is a cryoballoon, only a portion of an external surface of the cryoballoon is arranged to be cooled, and preferably, wherein the portion of the external surface is at a distal end of the cryoballoon.

Clause 27. The occlusion device according to any one of clauses 23 to 26, wherein the second catheter is a steerable delivery catheter and optionally, a distal end of the second catheter comprises a nozzle for accurately dispelling adhesive.

Clause 28. The occlusion device according to any one of clauses 23 to 27, wherein the adhesive is a bioglue.

1. An occlusion device for occluding a left atrial appendage, the device comprising:
a first catheter with a proximal end and a distal end, the distal end arranged to be positioned at an opening of the left atrial appendage;
a central lumen extending from a proximal portion to a distal portion of the first catheter;
a deployable member arranged at the distal end of the first catheter; and
a second catheter within the central lumen of the first catheter and arranged to deliver adhesive to the distal end of the first catheter.

2. The occlusion device according to claim 1, further comprising a pressure sensor for sensing the pressure in the deployable member at the distal end of the catheter,
wherein the pressure sensor is arranged to send a signal to a control system if a measured pressure value crosses a predetermined threshold.

3. The occlusion device according to claim 1, wherein the deployable member is a cryocooled member.

4. The occlusion device according to claim 3, wherein the cryocooled member is a cryocooled tip or a cryoballoon.

5. The occlusion device according to claim 4, wherein when the deployable member is a cryoballoon, and
wherein only a portion of an external surface of the cryoballoon is arranged to be cooled.

6. The occlusion device of claim 5, wherein the portion of the external surface is at a distal end of the cryoballoon.

7. The occlusion device according to claim 1, wherein the second catheter is a steerable delivery catheter.

8. The occlusion device according to claim 1, wherein a distal end of the second catheter comprises a nozzle configured to accurately dispelling adhesive.

9. The occlusion device according to claim 1, wherein the adhesive is a bioglue.

10. The occlusion device according to claim 1, wherein the adhesive comprises radiopaque markers.

11. A method of occluding a left atrial appendage, the method comprising:
positioning a catheter adjacent to an opening of the left atrial appendage, the catheter comprising an elongate tubular body and a central lumen that extends from a proximal end to a distal end of the catheter, the catheter comprising a deployable member arranged at the distal end of the catheter;
advancing the distal end of the catheter through the opening and into the left atrial appendage;
contacting a wall of the left atrial appendage with the deployable member and adhering the deployable member with the wall of the left atrial appendage;
retracting the catheter proximally to move the wall from an original position to a second position, wherein in the second position, the left atrial appendage wall is at least partially inverted thereby reducing the internal volume of the left atrial appendage; and
inserting adhesive into the left atrial appendage to secure the left atrial appendage in the second position.

12. The method according to claim 11, wherein partially inverting the left atrial appendage wall comprises forming a peak with the left atrial appendage wall with an annular pocket.

13. The method according to claim 11, wherein partially inverting the left atrial appendage wall comprises invaginating the left atrial appendage.

14. The method according to claim 11, wherein the deployable member is a cryocooled member, the method further comprising cooling the cryocooled member before adhering the cryocooled member with the wall of the left atrial appendage.

15. The method of according to claim 14, wherein the cryocooled member is a cryocooled tip of the catheter or a cryoballoon.

16. The method according to claim 15, wherein the cryocooled member is a cryoballoon, and
wherein the cryoballoon is collapsed when the catheter is positioned adjacent to the opening of the left atrial appendage and expanded before adhering the cryoballoon with the wall of the left atrial appendage.

17. The method according to claim 16, the method further comprising increasing an internal pressure of the cryoballoon before proximally retracting the left atrial appendage wall with the deployable member.

18. The method according to claim 11, the method further comprising incorporating radiopaque markers in the adhesive.

19. The method according claim 11, the method further comprises curing the adhesive before the deployable member is unadhered from the left atrial appendage wall.

20. The method according to claim 11, wherein the catheter is a first catheter, and wherein inserting the adhesive comprises:
inserting a distal end of a second catheter towards the opening of the left atrial appendage; and
delivering adhesive in the at least partially inverted portion of the left atrial appendage.
